(19) United States
(12) Patent Application Publication
(10) Pub. No.: US 2025/0302824 A1
(43) Pub. Date: Oct. 2, 2025

(54) IN SITU READY-TO-USE INJECTION FORMULATIONS OF POSACONAZOLE FREE OF CYCLODEXTRIN AND ITS DERIVATIVES

(71) Applicant: Hefei Cosource Pharmaceuticals Co., Ltd, Hefei (CN)

(72) Inventors: Yihua WANG, Hefei (CN); Dengjun CHEN, Hefei (CN); Yuting JIN, Hefei (CN); Jingnan DING, Hefei (CN); Jiandong LIAO, Hefei (CN); Zhiyun LI, Hefei (CN); Chengjing LU, Hefei (CN); Qianqian HU, Hefei (CN)

(21) Appl. No.: 19/094,752

(22) Filed: Mar. 28, 2025

(30) Foreign Application Priority Data
Mar. 29, 2024 (CN) 202410382768.3

(51) Int. Cl. A61K 31/496; A61K 9/00; A61K 9/08; A61K 9/51; A61K 47/10; A61K 47/22

(52) U.S. Cl. CPC A61K 31/496; A61K 9/0019; A61K 9/08; A61K 9/5123; A61K 47/10; A61K 47/22

(57) ABSTRACT

The present disclosure relates to an in situ ready-to-use injection formulation of posaconazole free of cyclodextrin and derivatives of cyclodextrin, which can be formulated in situ as a nanosuspension injection of posaconazole by a simple dilution operation during clinical use. The formulation has no adverse effects on the renal function of patients, no extreme pH, and low vascular irritation, and can be administrated without the need for central venous cannulation during clinical use. The present disclosure also relates to a method for preparing the formulation and the use of the formulation in the treatment and prevention of fungal infections.

### CROSS-REFERENCE TO RELATED APPLICATIONS

The present application claims the benefit and priority to the Chinese patent application No. 202410382768.3 filed before the China national intellectual property administration on Mar. 29, 2024, the content of which is incorporated herein by reference in its entirety.

### TECHNICAL FIELD

The present disclosure relates to the field of pharmaceutical formulations, and more specifically, to a pharmaceutical formulation of posaconazole for antifungal infections and a preparation method thereof.

### BACKGROUND

Posaconazole belongs to the triazole antifungal agents, and is represented by the following chemical structural formula:

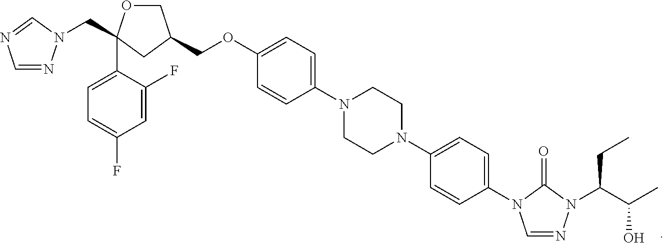 [SMILES: CC[C@@H]([C@H](C)O)n1ncn(-c2ccc(N3CCN(c4ccc(OC[C@@H]5CO[C@@](Cn6cncn6)(c6ccc(F)cc6F)C5)cc4)CC3)cc2)c1=O]

Posaconazole inhibits the synthesis of ergosterol by inhibiting CYP450-dependent 14α-demethylase, leading to disorders in biosynthesis of fungal cell membranes and changes in cell membrane permeability, thereby inhibiting fungal growth. The antimicrobial spectrum of posaconazole includes common Candida, Cryptococcus, and Aspergillus, as well as rare fungi such as Mucor, Coccidioides, Fusarium, and Scedosporium Prolificans. Posaconazole has a broader antimicrobial spectrum, stronger antimicrobial activity, and good tolerability, and has a wide range of applications in the prophylaxis and treatment of invasive fungal diseases (IFDs), compared with echinocandins and other triazole drugs. An oral suspension formulation of posaconazole was approved by the U.S. Food and Drug Administration and marketed in 2005, followed by the emergence of enteric-coated tablets and injections of posaconazole.

Posaconazole is a weakly alkaline drug with poor water-solubility, and has a solubility of less than 1 μg/ml in neutral and alkaline solutions, and a slightly increased solubility under acidic conditions (a solubility of 3 μg/ml at pH 3 and 0.8 mg/ml at pH 1). However, the daily dosage of posaconazole is 100 mg or more in clinical use, and therefore, the low solubility of posaconazole limits the development of its formulations.

In order to address its low solubility, a posaconazole injection containing about 40% sulfobutyl ether-β-cyclodextrin has been marketed. The main adverse effect of cyclodextrin is nephrotoxicity, which can cause vacuolar lesions at the distal end of renal tubules and even necrosis of renal tubular cells. Patients with renal insufficiency are susceptible to accumulation of cyclodextrin when using a pharmaceutical formulation containing cyclodextrin, which increases renal toxicity and further aggravates renal damage. Therefore, a pharmaceutical formulation containing cyclodextrin is used with caution or even contraindicated in patients with renal insufficiency in clinical practice. However, posaconazole is mostly applied in immunodeficient population or tumour patients. These individuals are prone to renal dysfunction when receiving chemotherapy and immunotherapy. For example, cisplatin interacts with renal proximal convoluted tubular cells during renal excretion in its prototype form, causing tubular necrosis. For another example, panitumumab interferes with the movement of TRPM6 towards the apical membrane of renal distal convoluted tubules, reducing magnesium ion reabsorption, leading to hypomagnesemia, and damaging the proximal renal tubules. Therefore, posaconazole injections containing cyclodextrin or its derivatives are clinically restricted for administration to patients with renal insufficiency.

In addition, the marketed posaconazole injection specifies in the instructions that it should be administered through central venous access, including central venous catheters or peripherally inserted central catheters, and each administration should be a slow intravenous infusion for 90 minutes or more. This is due to the fact that the marketed posaconazole injection has a low pH value (about pH 2.6) and strong vascular irritation, and is prone to cause adverse effects at the infusion site.

Central venous access is an access device for the purpose of clinical diagnosis and treatment, in which the tip of a catheter inserted through venipuncture is located in the central vein. Central venous access includes Central Venous Catheters (CVCs) inserted through the internal jugular vein, subclavian vein, and femoral vein, Peripherally Inserted Central Catheters (PICCs) via venipuncture through the basilic vein of the upper limb, the median cubital vein, the cephalic vein, and the brachial vein, as well as a closed infusion device, Implantable Venous Access Port (PORT), implanted completely into the body through the internal jugular vein or subclavian vein, etc. Administration through central venous access has high requirements for aseptic operations and requires specially trained medical personnel to carry out, with poor patient comfort, and high diagnostic and therapeutic costs, and inadvertent puncture of nerves, arteries, and lymph may occur during venipuncture and indwelling, resulting in complications such as bleeding, exudation, infections, phlebitis, thrombosis, catheter displacement, catheter misplacement, catheter obstruction and disconnection. Some complications may cause irreversible damages, such as nerve damage, thrombosis, catheter detachment into the pulmonary artery, etc.

Therefore, there is an urgent need for in situ ready-to-use injection formulations of posaconazole that are free of cyclodextrin and its derivatives. In particular, there is an urgent need for in situ ready-to-use injection formulations of posaconazole that are free of cyclodextrin and its derivatives and do not require administration through the central venous access.

### SUMMARY OF INVENTION

In one aspect, the present disclosure provides a pharmaceutical composition comprising posaconazole or a pharmaceutically acceptable salt thereof, a good solvent, a stabilizer, a surfactant, a nanocrystal inducer and optionally a pH regulator, wherein the pharmaceutical composition is free of cyclodextrin and derivatives of cyclodextrin.

In another aspect, the present disclosure provides a method for treating and preventing fungal infections in a patient, comprising administering the pharmaceutical composition of the present disclosure to the patient.

### BRIEF DESCRIPTION OF DRAWINGS

FIG. 1 shows the mean drug concentration-time profile in plasma for the pharmaceutical formulation of Example 5 and the marketed posaconazole injection.

FIG. 2 shows the mean drug concentration-time profile in the heart for the pharmaceutical formulation of Example 5 and the marketed posaconazole injection.

FIG. 3 shows the mean drug concentration-time profile in the liver for the pharmaceutical formulation of Example 5 and the marketed posaconazole injection.

FIG. 4 shows the mean drug concentration-time profile in the spleen for the pharmaceutical formulation of Example 5 and the marketed posaconazole injection.

FIG. 5 shows the mean drug concentration-time profile in the lung for the pharmaceutical formulation of Example 5 and the marketed posaconazole injection.

FIG. 6 shows the mean drug concentration-time profile in the kidney for the pharmaceutical formulation of Example 5 and the marketed posaconazole injection.

FIG. 7 shows the mean drug concentration-time profiles in plasma and tissues for the pharmaceutical formulation of Example 5.

FIG. 8 shows a histogram of the distribution in plasma and tissues for the pharmaceutical formulation of Example 5 and the marketed posaconazole injection.

FIG. 9A and FIG. 9B show the results of in vitro hemolysis experiments of the pharmaceutical formulation of Example 5 and the marketed posaconazole injection after (A) 3-hour exposure and (B) 3-hour exposure followed by centrifugation.

FIG. 10 shows the operation procedures of the in situ ready-to-use pharmaceutical composition of posaconazole of the present disclosure in clinical practice.

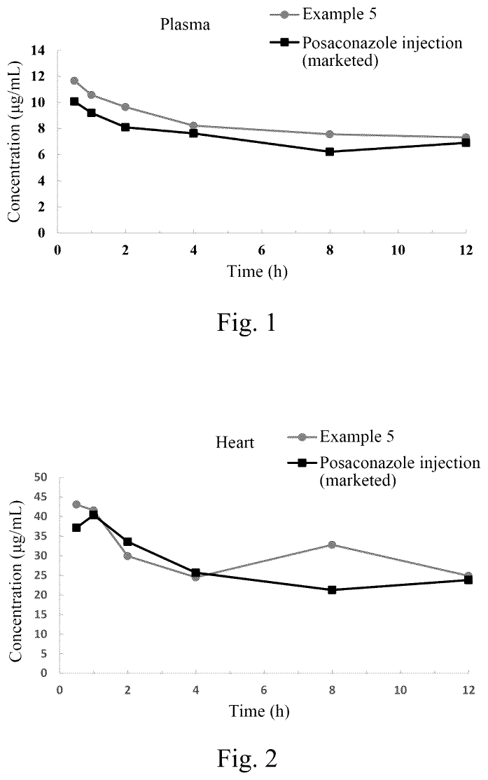

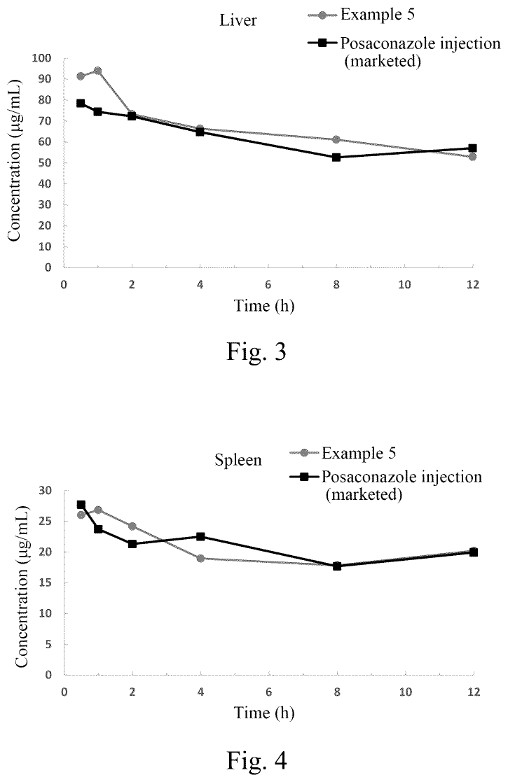

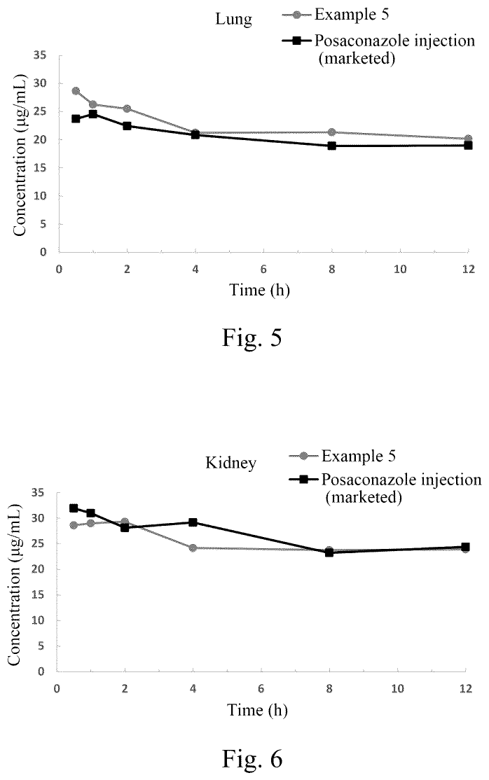

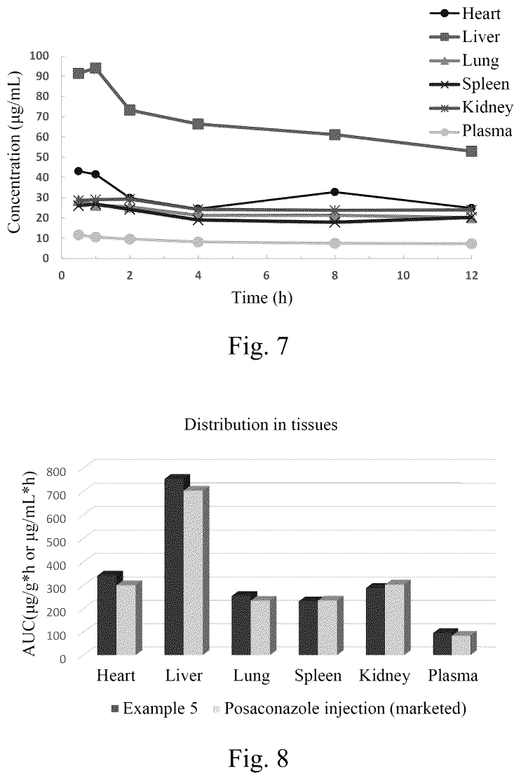

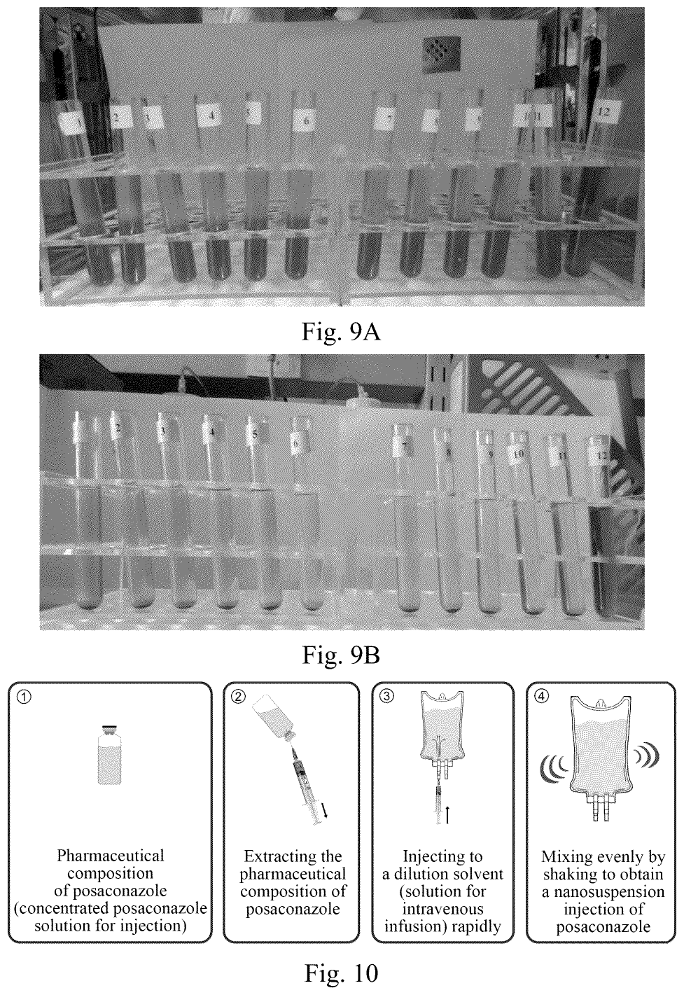

### DESCRIPTION OF EMBODIMENTS

In one aspect, the present disclosure provides a pharmaceutical composition comprising posaconazole or a pharmaceutically acceptable salt thereof, a good solvent, a stabilizer, a surfactant, a nanocrystal inducer and optionally a pH regulator, wherein the pharmaceutical composition is free of cyclodextrin and derivatives of cyclodextrin.

In some embodiments, the pharmaceutical composition is an intravenous injection formulation, which is administered for example through intravenous drip, intravenous infusion, intravenous injection pump, intravenous slow bolus injection, or other routes.

In some embodiments, the good solvent is a good solvent suitable for posaconazole. In some embodiments, the good solvent is selected from the group consisting of anhydrous ethanol, propylene glycol, polyethylene glycol, dimethyl sulfoxide, N-methylpyrrolidone, N,N-dimethylacetamide, N,N-dimethylformamide, and any combination thereof. In some embodiments, the good solvent is selected from the group consisting of a mixture of anhydrous ethanol and polyethylene glycol, a mixture of propylene glycol and polyethylene glycol, dimethyl sulfoxide, N-methylpyrrolidone, N,N-dimethylacetamide, and N,N-dimethylformamide. In some embodiments, the good solvent is selected from the group consisting of N,N-dimethylacetamide, N,N-dimethylformamide, dimethyl sulfoxide, a mixture of propylene glycol and polyethylene glycol, and a mixture of anhydrous ethanol and polyethylene glycol. In some embodiments, the good solvent is selected from a mixture of anhydrous ethanol and polyethylene glycol 400, a mixture of anhydrous ethanol and polyethylene glycol 300, a mixture of anhydrous ethanol and polyethylene glycol 200, a mixture of propylene glycol and polyethylene glycol 400, a mixture of propylene glycol and polyethylene glycol 300, or a mixture of propylene glycol and polyethylene glycol 200. In some embodiments, the good solvent is selected from a mixture of anhydrous ethanol and polyethylene glycol 400, a mixture of anhydrous ethanol and polyethylene glycol 300, or a mixture of anhydrous ethanol and polyethylene glycol 200. In some embodiments, a volume ratio of anhydrous ethanol or propylene glycol to polyethylene glycol may range from about 1:9 to about 1:1, for example, about 1:8, 1:7, 1:6, 1:5, 1:4, 1:3 or 1:2.

The pharmaceutical composition of the present disclosure further comprises the stabilizer to make posaconazole more stable in the good solvent. In some embodiments, the stabilizer is selected from the group consisting of ascorbyl palmitate, sodium metabisulfite, α-tocopherol, butylated droxyanisole, butylated hydroxytoluene, methionine, proline, glycine, and any combination thereof. In some embodiments, the stabilizer is selected from the group consisting of ascorbyl palmitate, α-tocopherol, butylated droxyanisole, butylated hydroxytoluene, methionine, proline, glycine, and any combination thereof. In some embodiments, the stabilizer is selected from the group consisting of methionine, α-tocopherol, ascorbyl palmitate, butylated droxyanisole, butylated hydroxytoluene, and any combination thereof. In some embodiments, the stabilizer is selected from the group consisting of α-tocopherol, ascorbyl palmitate, butylated droxyanisole, butylated hydroxytoluene, and any combination thereof. In some embodiments, the stabilizer is selected from the group consisting of α-tocopherol, ascorbyl palmitate, a combinaiton of α-tocopherol and ascorbyl palmitate, and a combination of butylated droxyanisole and butylated hydroxytoluene.

The pharmaceutical composition of the present disclosure further comprises the surfactant, wherein the surfactant is selected from the group consisting of glycocholic acid, glycocholate (such as sodium salt, potassium salt, ammonium salt, etc.), deoxycholic acid, deoxycholate (such as sodium salt, potassium salt, ammonium salt, etc.), tauroursodeoxycholic acid, tauroursodeoxycholate (such as sodium salt, potassium salt, ammonium salt, etc.), taurocholic acid, taurocholate (such as sodium salt, potassium salt, ammonium salt, etc.), taurodeoxycholic acid, taurodeoxycholate (such as sodium salt, potassium salt, ammonium salt, etc.), polyoxyl 15 hydroxystearate, polyethylene glycol vitamin E succinate, and any combination thereof. In some embodiments, the surfactant is selected from the group consisting of polyethylene glycol vitamin E succinate, deoxycholic acid, deoxycholate, glycocholic acid, glycocholate, taurodeoxycholic acid, taurodeoxycholate, and any combination thereof. In some embodiments, the surfactant is selected from one or two of glycocholic acid, sodium glycocholate, and polyethylene glycol vitamin E succinate.

The pharmaceutical composition of the present disclosure further comprises the nanocrystal inducer, wherein the nanocrystal inducer is selected from the group consisting of pamoic acid, hydroxynaphthoic acid, propyl gallate, benzoic acid, and any combination thereof. In some embodiments, the nanocrystal inducer is selected from pamoic acid and/or hydroxynaphthoic acid. In some embodiments, examples of hydroxynaphthoic acid include, but are not limited to, 2-hydroxy-1-naphthoic acid, 3-hydroxy-1-naphthoic acid, 4-hydroxy-1-naphthoic acid, 5-hydroxy-1-naphthoic acid, 6-hydroxy-1-naphthoic acid, 1-hydroxy-2-naphthoic acid, 2-hydroxy-3-naphthoic acid, and 8-hydroxy-2-naphthoic acid. In some embodiments, the nanocrystal inducer is selected from pamoic acid, 3-hydroxy-1-naphthoic acid, and 2-hydroxy-3-naphthoic acid. In some embodiments, the nanocrystal inducer is pamoic acid.

The inventors of the present disclosure have found that the addition of the surfactant and the nanocrystal inducer helps to formulate a nanosuspension in situ from the resulting pharmaceutical composition upon dilution for clinical applications. In some embodiments, the particle size of solid particles in the nanosuspension ranges from about 50 nm to about 300 nm, and the nanosuspension can be stably stored at room temperature for at least 12 hours. In some embodiments, the particle size of solid particles in the nanosuspension ranges from about 50 nm to about 200 nm, and the nanosuspension can be stably stored at room temperature for at least 12 hours, or even at room temperature for at least 24 hours.

In some embodiments, the pharmaceutical composition of the present disclosure has a pH of about 3.5 to about 8.0 after dilution. In some embodiments, the pharmaceutical composition of the present disclosure has a pH of about 4.0 to about 6.0 after dilution.

In some embodiments, the pharmaceutical composition of the present disclosure may further comprise the pH regulator to increase the solubility of the nanocrystal inducer such as pamoic acid. In some embodiments, the pH regulator is a commonly-used alkaline regulator in the art, as long as the addition of the pH regulator can increase the solubility of the nanocrystal inducer such as pamoic acid. Examples of the pH regulator include, but are not limited to, sodium hydroxide, potassium hydroxide, tromethamine, meglumine, and any combination thereof. In some embodiments, the pH regulator is selected from the group consisting of sodium hydroxide and potassium hydroxide.

In some embodiments, the pharmaceutical composition comprises about 1 g to about 10 g of posaconazole in the form of a free base, about 0.01 g to about 1.0 g of the stabilizer, about 0.1 g to about 7.0 g of the surfactant, about 0.2 g to about 4.0 g of the nanocrystal inducer, and about 0 g to about 1.0 g of the pH regulator, based on 100 ml of a total volume of the good solvent. In some embodiments, the pharmaceutical composition comprises about 2.0 g to about 3.0 g of posaconazole in the form of a free base, about 0.1 g to about 0.2 g of the stabilizer, about 0.2 g to about 3.0 g of the surfactant, about 0.2 g to about 2.0 g of the nanocrystal inducer, and about 0.1 g to about 0.5 g of the pH regulator, based on 100 ml of a total volume of the good solvent.

In some embodiments, the pharmaceutical composition comprises about 1 g to about 10 g of posaconazole in the form of a free base, about 0.01 g to about 1.0 g of α-tocopherol or a mixture of α-tocopherol and ascorbyl palmitate, about 0.1 g to about 5.0 g of glycocholic acid or sodium glycocholate, about 0.2 g to about 4.0 g of pamoic acid or hydroxynaphthoic acid, and about 0 g to about 1.0 g of sodium hydroxide or potassium hydroxide, based on 100 ml of a total volume of a mixture of anhydrous ethanol and PEG. In some embodiments, the pharmaceutical composition comprises about 2.0 g to about 3.0 g of posaconazole in the form of a free base, about 0.1 g to about 0.2 g of α-tocopherol or a mixture of α-tocopherol and ascorbyl palmitate, about 0.2 g to about 3.0 g of glycocholic acid or sodium glycocholate, about 0.2 g to about 2.0 g of pamoic acid or hydroxynaphthoic acid, and about 0.1 g to about 0.5 g of sodium hydroxide or potassium hydroxide, based on 100 ml of a total volume of a mixture of anhydrous ethanol and PEG.

In some embodiments, the concentration of posaconazole in the form of a free base in the pharmaceutical composition ranges from about 10 mg/ml to about 100 mg/ml, or from about 20 mg/ml to about 30 mg/ml, prior to the addition of an antisolvent. In some embodiments, the concentration of posaconazole in the form of a free base in the pharmaceutical composition ranges from about 0.5 mg/ml to about 5 mg/ml, or from about 1 mg/ml to about 2 mg/ml, after the addition of an antisolvent.

The pharmaceutical composition of the present disclosure is diluted with an antisolvent before administration to the patient. The antisolvent is a clinically available dilution solvent for intravenous infusion, and selected from the group consisting of sterilized water for injection, 5% aqueous glucose solution, 10% aqueous glucose solution, 0.45% aqueous sodium chloride solution, 0.9% aqueous sodium chloride solution, 5% aqueous glucose and 0.45% aqueous sodium chloride solution, 5% aqueous glucose and 0.9% aqueous sodium chloride solution, and 5% aqueous glucose and 20 mEq aqueous potassium chloride solution.

In some embodiments, the pharmaceutical composition of the present disclosure is a clear, transparent and true solution. The pharmaceutical composition of the present disclosure is diluted by addition to the antisolvent immediately prior to use, to obtain a nanosuspension injection.

In another aspect, the present disclosure provides a method for preparing the pharmaceutical composition, comprising adding posaconazole, the stabilizer, the surfactant, the nanocrystal inducer, and optionally the pH regulator to the good solvent to dissolve the components. If necessary, the components may be dissolved by heating.

In some embodiments, the pharmaceutical composition of the present disclosure can be protected with nitrogen during preparation and storage.

In still another aspect, the present disclosure provides a method for treating and preventing fungal infections in a patient, comprising administering the pharmaceutical composition of the present disclosure to the patient. In some embodiments, the fungal infection is selected from the group consisting of oropharyngeal or esophageal candidiasis; refractory oropharyngeal or esophageal candidiasis; invasive aspergillosis, candidiasis, fusaridiosis, scedosporiosis, infections caused by dimorphic fungi, zygomycosis, and invasive infections caused by rare fungi and yeasts; invasive fungal diseases in patients who are not responsive or intolerant to other therapies; candidiasis and invasive mycotic infections in patients undergoing intensive chemotherapies and/or radiotherapies due to hematological malignancies, bone marrow or peripheral stem cell transplantation pretreatment regimens, and in patients receiving combined immunosuppressive therapies for the treatment of acute or chronic graft-versus-host disease or for the prevention of solid organ transplant rejection; Chagas's disease; and leishmaniasis.

The pharmaceutical composition of the present disclosure has one or more of the following advantages: (1) it is free of cyclodextrin and derivatives of cyclodextrin, and can be used in patients with renal insufficiency; (2) it has a pH value closer to the physiological pH, reducing irritation to the injection site and adverse effects, and can be administrated clinically without the need to use the central venous access, improving the convenience of clinical administration and enhancing the patient's compliance; (3) it enables the administration to patients who cannot be administered oral dosage forms, such as patients with dysphagia or unconsciousness; (4) it can be formulated in situ as a nanosuspension injection through simple dilution during clinical use; (5) it has high stability with a stable particle size distribution, and no significant change in particle size over time, and/or the formulation remains stable when stored under accelerated conditions and room temperature conditions, with a low content of maximum single impurity and/or a low content of total impurity; (6) it can be stored at room temperature (10° C. to 30° C.), making storage and transportation more convenient; (7) it has similar pharmacokinetic parameters to the marketed formulation (such as the posaconazole injection) at the same dosage, with similar bioavailability and distribution in tissues; and (8) it has no hemolytic or coagulant effect on the red blood cells of rabbits.

### EXAMPLES

The present disclosure will be described in further details below in conjunction with specific examples. These examples may be modified to obtain other embodiments without departing from the scope or spirit of the present disclosure. Therefore, the following examples are non-limitative.

Unless otherwise specified, all numbers used in the Specification and Claims to represent feature sizes, quantities, and physicochemical properties should be understood as being modified by the term “about” in all cases. Therefore, unless otherwise stated to the contrary, the numerical parameters listed in the foregoing Specification and the appended claims are approximations, and those skilled in the art can utilize the teachings disclosed herein to seek the desired characteristics, and appropriately change these approximations. The use of a numerical range represented by endpoints includes all numbers within said range and any range within said range, for example, 1 to 5 includes 1, 1.1, 1.3, 1.5, 2, 2.75, 3, 3.80, 4, and 5, etc.

The drugs or reagents used in the present disclosure are all conventional commercially-available products, unless otherwise specified.

General preparation method: the components listed in the table below are weighed, and added to the solvent with thorough stirring and dispersion, and if necessary, they can be placed in a water bath at 40-60° C. with thorough stirring to dissolve each component to obtain a clear and transparent solution. Before use, the solution is diluted with the antisolvent to the concentration of posaconazole in the form of a free base of about 1 mg/ml to about 2 mg/ml.

### Comparative Example 1

| | Weighed amount | Weight to volume |
|---|---|---|
| Components | (g) | percentage (W/V %) |
| Posaconazole | 1.5 | 3.00 |
| Glycocholic acid | 0.75 | 1.50 |
| Propyl hydroxybenzoate | 0.4 | 0.80 |
| (crystallization inducer) | | |
| Sodium hydroxide | 0.12 | 0.24 |
| α-tocopherol | 0.05 | 0.10 |
| Anhydrous ethanol | 10 | |
| Polyethylene glycol 400 | 40 | |

### Comparative Example 2

| | Weighed amount | Weight to volume |
|---|---|---|
| Components | (g) | percentage (W/V %) |
| Posaconazole | 1.5 | 3.00 |
| Poloxamer P188 (surfactant) | 0.5 | 1.00 |
| Pamoic acid | 0.4 | 0.80 |
| Sodium hydroxide | 0.12 | 0.24 |
| α-tocopherol | 0.05 | 0.10 |
| Anhydrous ethanol | 10 | |
| Polyethylene glycol 400 | 40 | |

### Comparative Example 3

| | Weighed amount | Weight to volume |
|---|---|---|
| Components | (g) | percentage (W/V %) |
| Posaconazole | 1.5 | 3.00 |
| Sodium deoxycholate | 0.75 | 1.50 |
| Pamoic acid | 0.4 | 0.80 |
| Sodium hydroxide | 0.05 | 0.10 |
| Thioglycerol (stabilizer) | 0.05 | 0.10 |
| Anhydrous ethanol | 10 | |
| Polyethylene glycol 400 | 40 | |

### Comparative Example 4

| | Weighed amount | Weight to volume |
|---|---|---|
| Components | (g) | percentage (W/V %) |
| Posaconazole | 1.5 | 3.00 |
| Sodium deoxycholate | 0.75 | 1.50 |
| Pamoic acid | 0.4 | 0.80 |
| Sodium hydroxide | 0.05 | 0.10 |
| Anhydrous ethanol | 10 | |
| Polyethylene glycol 400 | 40 | |

### Comparative Example 5

| | Weighed amount | Weight to volume |
|---|---|---|
| Components | (g) | percentage (W/V %) |
| Posaconazole | 1.5 | 3.00 |
| Glycocholic acid | 0.75 | 1.50 |
| Pamoic acid | 0.5 | 1.00 |
| Sodium hydroxide | 0.25 | 0.50 |
| Benzyl alcohol (solvent) | 10 | |
| Polyethylene glycol 200 | 40 | |

### Example 1

| | Weighed amount | Weight to volume |
|---|---|---|
| Components | (g) | percentage (W/V %) |
| Posaconazole | 3 | 10.0 |
| Polyethylene glycol vitamin E | 1.5 | 5.0 |
| succinate | | |
| 3-hydroxy-1-naphthoic acid | 0.3 | 1.0 |
| Methionine | 0.15 | 0.5 |
| N,N-dimethylacetamide | 30 | |

### Example 2

| | | Weight to volume |
|---|---|---|
| Components | Weighed amount (g) | percentage (W/V %) |
| Posaconazole | 3 | 10.0 |
| Sodium deoxycholate | 1 | 3.3 |
| Pamoic acid | 0.3 | 1.0 |
| Methionine | 0.15 | 0.5 |
| N,N-dimethylformamide | 30 | |

### Example 3

| | | Weight to volume |
|---|---|---|
| Components | Weighed amount (g) | percentage (W/V %) |
| Posaconazole | 3 | 6.0 |
| Sodium taurodeoxycholate | 1 | 2.0 |
| Propyl gallate | 0.3 | 0.6 |
| Proline | 0.15 | 0.3 |
| Dimethyl sulfoxide | 50 | |

### Example 4

| | | Weight to volume |
|---|---|---|
| Components | Weighed amount (g) | percentage (W/V %) |
| Posaconazole | 1.5 | 3.00 |
| Glycocholic acid | 1 | 2.00 |
| Benzoic acid | 0.5 | 1.00 |
| Sodium hydroxide | 0.02 | 0.04 |
| Glycine | 0.5 | 1.00 |
| Anhydrous ethanol | 5 | |
| Polyethylene glycol 200 | 45 | |

### Example 5

| | | Weight to volume |
|---|---|---|
| Components | Weighed amount ( g) | percentage (W/V %) |
| Posaconazole | 1.5 | 3.0 |
| Glycocholic acid | 1 | 2.0 |
| Pamoic acid | 1 | 2.0 |
| Sodium hydroxide | 0.25 | 0.5 |
| α-tocopherol | 0.05 | 0.10 |
| Anhydrous ethanol | 5 | |
| Polyethylene glycol 400 | 45 | |

### Example 6

| | | Weight to volume |
|---|---|---|
| Components | Weighed amount (g) | percentage (W/V %) |
| Posaconazole | 1.5 | 3.0 |
| Glycocholic acid | 2 | 4.0 |
| Pamoic acid | 1 | 2.0 |
| Sodium hydroxide | 0.25 | 0.5 |
| α-tocopherol | 0.05 | 0.10 |
| Anhydrous ethanol | 5 | |
| Polyethylene glycol 300 | 45 | |

### Example 7

| | Weighed amount | Weight to volume |
|---|---|---|
| Components | 0 | percentage (W/V %) |
| Posaconazole | 1.5 | 3.0 |
| Glycocholic acid | 0.05 | 0.1 |
| Pamoic acid | 1 | 2.0 |
| Sodium hydroxide | 0.25 | 0.5 |
| α-tocopherol | 0.05 | 0.10 |
| Anhydrous ethanol | 5 | |
| Polyethylene glycol 300 | 45 | |

### Example 8

| | | Weight to volume |
|---|---|---|
| Components | Weighed amount (g) | percentage (W/V %) |
| Posaconazole | 1.5 | 3.0 |
| Glycocholic acid | 0.5 | 1.0 |
| Pamoic acid | 2 | 4.0 |
| Sodium hydroxide | 0.25 | 0.5 |
| α-tocopherol | 0.05 | 0.10 |
| Anhydrous ethanol | 5 | |
| Polyethylene glycol 300 | 45 | |

### Example 9

| | | Weight to volume |
|---|---|---|
| Components | Weighed amount (g) | percentage (W/V %) |
| Posaconazole | 1.5 | 3.0 |
| Glycocholic acid | 2 | 4.0 |
| Pamoic acid | 0.5 | 1.0 |
| Sodium hydroxide | 0.2 | 0.4 |
| α-tocopherol | 0.05 | 0.10 |
| Anhydrous ethanol | 5 | |
| Polyethylene glycol 300 | 45 | |

### Example 10

| | | Weight to volume |
|---|---|---|
| Components | Weighed amount (g) | percentage (W/V %) |
| Posaconazole | 1.5 | 3.0 |
| Glycocholic acid | 1 | 2.0 |
| Pamoic acid | 0.5 | 1.0 |
| Potassium hydroxide | 0.15 | 0.3 |
| α-tocopherol | 0.05 | 0.10 |
| Propylene glycol | 5 | |
| Polyethylene glycol 300 | 45 | |

### Example 11

| | | Weight to volume |
|---|---|---|
| Components | Weighed amount (g) | percentage (W/V %) |
| Posaconazole | 1 | 2.0 |
| Glycocholic acid | 1 | 2.0 |
| Pamoic acid | 0.5 | 1.0 |
| Meglumine | 0.5 | 1.0 |
| α-tocopherol | 0.05 | 0.10 |
| Anhydrous ethanol | 5 | |
| Polyethylene glycol 400 | 45 | |

### Example 12

| | | Weight to volume |
|---|---|---|
| Components | Weighed amount (g) | percentage (W/V %) |
| Posaconazole | 1 | 2.0 |
| Glycocholic acid | 1 | 2.0 |
| Pamoic acid | 0.5 | 1.0 |
| Sodium hydroxide | 0.4 | 0.8 |
| α-tocopherol | 0.05 | 0.10 |
| Anhydrous ethanol | 5 | |
| Polyethylene glycol 400 | 45 | |

### Example 13

| | | Weight to volume |
|---|---|---|
| Components | Weighed amount (g) | percentage (W/V %) |
| Posaconazole | 1.5 | 3.0 |
| Glycocholic acid | 0.75 | 1.5 |
| Pamoic acid | 0.4 | 0.8 |
| Sodium hydroxide | 0.12 | 0.24 |
| Ascorbyl palmitate | 0.05 | 0.10 |
| Anhydrous ethanol | 10 | |
| Polyethylene glycol 400 | 40 | |

### Example 14

| | | Weight to volume |
|---|---|---|
| Components | Weighed amount (g) | percentage (W/V %) |
| Posaconazole | 1.5 | 3.0 |
| Glycocholic acid | 0.75 | 1.5 |
| Pamoic acid | 0.4 | 0.8 |
| Sodium hydroxide | 0.12 | 0.24 |
| α-tocopherol | 0.025 | 0.05 |
| Ascorbyl palmitate | 0.025 | 0.05 |
| Anhydrous ethanol | 10 | |
| Polyethylene glycol 300 | 40 | |

### Example 15

| | | Weight to volume |
|---|---|---|
| Components | Weighed amount (g) | percentage (W/V %) |
| Posaconazole | 1.5 | 3.0 |
| Glycocholic acid | 0.75 | 1.5 |
| Pamoic acid | 0.4 | 0.8 |
| Sodium hydroxide | 0.12 | 0.24 |
| α-tocopherol | 0.025 | 0.05 |
| Ascorbyl palmitate | 0.025 | 0.05 |
| Anhydrous ethanol | 10 | |
| Polyethylene glycol 400 | 40 | |

### Example 16

| | | Weight to volume |
|---|---|---|
| Components | Weighed amount (g) | percentage (W/V %) |
| Posaconazole | 1.5 | 3.0 |
| Glycocholic acid | 0.75 | 1.5 |
| Pamoic acid | 0.4 | 0.8 |
| Sodium hydroxide | 0.12 | 0.24 |
| Butylated droxyanisole | 0.005 | 0.01 |
| Butylated hydroxytoluene | 0.005 | 0.01 |
| Anhydrous ethanol | 10 | |
| Polyethylene glycol 400 | 40 | |

### Example 17

| | Weighed | Weight to volume |
|---|---|---|
| Components | amount (g) | percentage (W/V %) |
| Posaconazole | 3 | 10.0 |
| Polyoxyl 15 hydroxystearate | 2 | 6.7 |
| Pamoic acid | 0.3 | 1.0 |
| Sodium metabisulfite | 0.05 | 0.17 |
| N-methylpyrrolidone | 30 | |

### Test Examples

### 1. Determination of Particle Size Distribution

Measurement method: the pharmaceutical formulations prepared in the comparative examples and examples were taken, and diluted with 5% glucose injection solution at room temperature to obtain nanosuspensions containing posaconazole at a concentration of 1.5 mg/ml, which were further diluted with ultrapure water by about 5 times, and placed in a sample cell. Then, the particle size (Z-average particle size) and polydispersity index (PDI) were measured using a BT-90Zeta nanoparticle size analyzer (Dandong Bettersize Instrument Co., Ltd.). The diluted nanosuspensions were left at room temperature for 2 h, 8 h, and 24 h, and the Z-average particle size and polydispersity index were measured at each time point to determine the stability of the particle size distribution. The measurement results were shown in Table 1.

TABLE 1

| | Placement | | Z-average particle | |
|---|---|---|---|---|
| Sample Name | time | | size (d · nm) | PDI |
| Comparative example 1 | 0 | h | 116.14 | 0.374 |
| | 2 | h | 3836.37 | 0.511 |
| Comparative example 2 | 0 | h | 190.43 | 0.393 |
| | 2 | h | 8982.76 | 0.47 |
| Comparative example 5 | 0 | h | 271.05 | 0.014 |
| | 2 | h | 656.64 | 0.521 |
| Example 1 | 0 | h | 128.78 | 0.099 |
| | 2 | h | 134.72 | 0.068 |
| | 8 | h | 136.93 | 0.056 |
| | 24 | h | 151.44 | 0.09 |
| Example 2 | 0 | h | 106.44 | 0.177 |
| | 2 | h | 101.45 | 0.147 |
| | 8 | h | 107.66 | 0.158 |
| | 24 | h | 108.55 | 0.142 |
| Example 3 | 0 | h | 99.47 | 0.111 |
| | 2 | h | 100.24 | 0.12 |
| | 8 | h | 98.47 | 0.159 |
| | 24 | h | 104.56 | 0.197 |
| Example 4 | 0 | h | 100.39 | 0.168 |
| | 2 | h | 100.56 | 0.194 |
| | 8 | h | 105.66 | 0.215 |
| | 24 | h | 103.68 | 0.186 |
| Example 5 | 0 | h | 96.63 | 0.119 |
| | 2 | h | 100.72 | 0.14 |
| | 8 | h | 99.96 | 0.145 |
| | 24 | h | 100.16 | 0.141 |
| Example 6 | 0 | h | 103.63 | 0.091 |
| | 2 | h | 105.75 | 0.097 |
| | 8 | h | 105.82 | 0.091 |
| | 24 | h | 106.97 | 0.104 |
| Example 9 | 0 | h | 98.57 | 0.058 |
| | 2 | h | 98.10 | 0.113 |
| | 8 | h | 101.25 | 0.098 |
| | 24 | h | 105.68 | 0.156 |
| Example 10 | 0 | h | 95.64 | 0.123 |
| | 2 | h | 95.7 | 0.097 |
| | 8 | h | 98.36 | 0.157 |
| | 24 | h | 99.2 | 0.168 |
| Example 13 | 0 | h | 95.87 | 0.15 |
| | 2 | h | 102.23 | 0.123 |
| | 8 | h | 100.42 | 0.159 |
| | 24 | h | 103.4 | 0.168 |
| Example 15 | 0 | h | 85.54 | 0.152 |
| | 2 | h | 88.62 | 0.131 |
| | 8 | h | 87.41 | 0.152 |
| | 24 | h | 86.15 | 0.16 |
| Example 17 | 0 | h | 90.15 | 0.168 |
| | 2 | h | 96.15 | 0.162 |
| | 8 | h | 89.36 | 0.113 |
| | 24 | h | 96.34 | 0.204 |

Conclusion: The pharmaceutical formulations in Comparative examples 1, 2, and 5 showed significant changes in Z-average particle size and polydispersity index after being placed at room temperature for 2 hours, demonstrating the poor stability of the particle size distribution. The pharmaceutical formulations of Examples 1-6, 9-10, 13, 15, and 17 of the present disclosure showed substantially no significant differences in Z-average particle size and polydispersity index after being placed at room temperature for 24 hours, demonstrating the good stability of the particle size distribution and more suitability for intravenous injection.

Using a procedure similar to the above procedure, the pharmaceutical formulation prepared in Example 15 was diluted with different antisolvents at room temperature, and left at room temperature for 0 h, 2 h, 8 h, 24 h, and 48 h. The Z-average particle size and polydispersity index were measured at each time point to determine the stability of the particle size distribution. The measurement results were shown in Table 2.

TABLE 2

| | Placement | | Z-average particle | |
|---|---|---|---|---|
| Sample Name | time | | size (d · nm) | PDI |
| Example 15- sodium chloride | 0 | h | 88.25 | 0.058 |
| injection solution | 2 | h | 84.77 | 0.088 |
| | 8 | h | 86.15 | 0.102 |
| | 24 | h | 88.45 | 0.098 |
| | 48 | h | 90.25 | 0.159 |
| Example 15-5% glucose | 0 | h | 88.54 | 0.058 |
| injection solution | 2 | h | 85.67 | 0.098 |
| | 8 | h | 90.24 | 0.088 |
| | 24 | h | 90.35 | 0.102 |
| | 48 | h | 92.89 | 0.201 |
| Example 15-10% glucose | 0 | h | 90.56 | 0.111 |
| injection solution | 2 | h | 90.25 | 0.105 |
| | 8 | h | 95.56 | 0.097 |
| | 24 | h | 89.88 | 0.156 |
| | 48 | h | 95.36 | 0.201 |
| Example 15-5% glucose and | 0 | h | 88.97 | 0.100 |
| 0.45% sodium chloride | 2 | h | 91.36 | 0.188 |
| aqueous solution | 8 | h | 97.51 | 0.154 |
| | 24 | h | 95.36 | 0.220 |
| | 48 | h | 95.14 | 0.228 |
| Example 15-water for | 0 | h | 88.78 | 0.047 |
| injection | 2 | h | 89.12 | 0.089 |
| | 8 | h | 86.56 | 0.105 |
| | 24 | h | 91.36 | 0.117 |
| | 48 | h | 93.56 | 0.145 |

Conclusion: the pharmaceutical formulation prepared in Example 15 showed no significant differences in Z-average particle size and polydispersity index after being diluted with different antisolvents and placed at room temperature for 48 h, indicating the good stability of the particle size distribution.

Using a procedure similar to the above procedure, the pharmaceutical formulation prepared in Example 15 was diluted with 5% glucose injection solution at 4° C., room temperature (about 20° C.), 30° C., 40° C., and 50° C., and then left for 0 h, 2 h, 8 h, 24 h, and 48 h at the respective temperatures. The Z-average particle size and polydispersity index were measured at each time point to determine the stability of the particle size distribution. The measurement results were shown in Table 3.

TABLE 3

| | Placement | | Z-average particle | |
|---|---|---|---|---|
| Sample Name | time | | size (d · nm) | PDI |
| Example 15-4° C. | 0 | h | 89.12 | 0.055 |
| | 2 | h | 88.45 | 0.047 |
| | 8 | h | 90.10 | 0.091 |
| | 24 | h | 90.11 | 0.102 |
| | 48 | h | 91.25 | 0.105 |
| Example 15- room temperature | 0 | h | 88.54 | 0.058 |
| (about 20° C.) | 2 | h | 85.67 | 0.098 |
| | 8 | h | 90.24 | 0.088 |
| | 24 | h | 90.35 | 0.102 |
| | 48 | h | 92.89 | 0.201 |
| Example 15-30° C. | 0 | h | 92.31 | 0.125 |
| | 2 | h | 90.12 | 0.201 |
| | 8 | h | 90.35 | 0.166 |
| | 24 | h | 93.45 | 0.205 |
| | 48 | h | 95.67 | 0.234 |
| Example 15-40° C. | 0 | h | 100.25 | 0.179 |
| | 2 | h | 103.45 | 0.114 |
| | 8 | h | 101.98 | 0.180 |
| | 24 | h | 112.35 | 0.201 |
| | 48 | h | 135.65 | 0.222 |
| Example 15-50° C. | 0 | h | 150.56 | 0.267 |
| | 2 | h | 148.23 | 0.301 |
| | 8 | h | 160.54 | 0.287 |
| | 24 | h | 200.68 | 0.408 |
| | 48 | h | 316.25 | 0.412 |

Conclusion: when the pharmaceutical formulation prepared in Example 15 was diluted and left at 30° C. or below, the nanosuspension obtained after dilution had solid particles with a smaller particle size, and the stability of the particle size and particle size distribution could be maintained within 48 hours; when the pharmaceutical formulation prepared in Example 15 was diluted and left at 40° C., the nanosuspension obtained after dilution showed no significant changes in the particle size and particle size distribution within 8 hours, but slight increase in the particle size after 8 hours; when the pharmaceutical formulation prepared in Example 15 was diluted and left at 50° C., the nanosuspension obtained after dilution had solid particles with a larger particle size, and the particle size had an obvious tendency to increase, which may be due to the increased movement and accelerated aggregation of the particles at higher temperatures, leading to an increase in the particle size.

### 2. Determination of pH Value

Measurement method: the pharmaceutical formulations prepared in the Examples and the marketed posaconazole injection (Jiangsu Aosaikang Pharmaceutical Co., Ltd., G220603) were taken and diluted with 5% glucose injection solution at room temperature to obtain nanosuspensions containing posaconazole at a concentration of 1.5 mg/ml, and the pH value was measured directly using a pH meter. The measurement results were shown in Table 4.

TABLE 4

| Sample name | pH value |
|---|---|
| Marked posaconazole injection formulation | 2.6 |
| Example 1 | 6.8 |
| Example 2 | 7.4 |
| Example 3 | 7.6 |
| Example 4 | 6.2 |
| Example 5 | 5.1 |
| Example 6 | 4.5 |
| Example 7 | 4.7 |
| Example 8 | 4.4 |
| Example 9 | 5.0 |
| Example 10 | 4.6 |
| Example 11 | 5.1 |
| Example 12 | 4.2 |
| Example 13 | 4.8 |
| Example 14 | 4.7 |
| Example 15 | 4.8 |
| Example 16 | 4.9 |
| Example 17 | 7.5 |

Conclusion: compared with the marketed posaconazole injection, the pharmaceutical formulations of Examples 1-17 of the present disclosure had pH values closer to the physiological pH.

### 3. Stability Study-Determination of Relevant Substances

Unless otherwise specified, impurities in posaconazole were determined using the following high-performance liquid chromatography conditions:

Relevant substances were determined by the high-performance liquid chromatography (General Rule 0512) specified in Part IV of the Chinese Pharmacopoeia, Edition 2020. Octadecylsilane-bonded silica gel was used as the filler; 0.05 mol/L ammonium acetate aqueous solution (pH was adjusted to 4.0 with acetic acid) was used as the mobile phase A; acetonitrile was used as the mobile phase B; and the gradient elution was carried out according to the following table, with a flow rate of 1.0 ml/min;

Gradient elution procedure:

| Time (min) | % B |
|---|---|
| 0.01 | 5.0 |
| 3.00 | 35.0 |
| 4.00 | 35.0 |
| 17.00 | 60.0 |
| 18.00 | 60.0 |
| 35.00 | 90.0 |
| 36.00 | 90.0 |
| 40.00 | 5.0 |
| 50.00 | 5.0 |

The detection wavelength was 262 nm. The theoretical plate number calculated based on the peak of posaconazole was not less than 3000. The separation degree between adjacent impurity peaks should meet the requirements. Another appropriate amount of the posaconazole injection was taken as a test sample, diluted with 50% acetonitrile to a solution containing 1.5 mg/ml of posaconazole and centrifuged, and the supernatant was taken and injected into the liquid chromatography, and the chromatogram was recorded. The content was calculated as the percentage of peak area using the area normalization method. The measurement results were shown in Table 5.

TABLE 5

| | | | | Maximum | Total |
|---|---|---|---|---|---|
| | Placement | Placement | | single impurity | impurities |
| Sample name | conditions | time | | (%) | (%) |
| Comparative | 0 day | | | 0.077 | 0.126 |
| example 3 | RT | 1 | month | 0.318 | 1.145 |
| | | 3 | months | 0.274 | 1.168 |
| | | 6 | months | 0.225 | 1.216 |
| | 40° C. | 1 | month | 0.485 | 1.67 |
| | | 3 | months | 0.552 | 1.894 |
| | | 6 | months | 0.813 | 2.258 |
| | 60° C. | 10 | days | 0.483 | 1.743 |
| | | 1 | month | 0.99 | 2.299 |
| Comparative | 0 day | | | 0.231 | 0.175 |
| example 4 | RT | 1 | month | 0.311 | 1.126 |
| | | 3 | months | 0.335 | 1.211 |
| | | 6 | months | 0.348 | 1.487 |
| | 40° C. | 1 | month | 0.676 | 2.24 |
| | | 3 | months | 0.732 | 2.242 |
| | | 6 | months | 1.012 | 2.513 |
| | 60° C. | 10 | days | 0.93 | 2.5 |
| | | 1 | month | 1.275 | 3.631 |
| Example 1 | 0 day | | | 0.022 | 0.045 |
| | RT | 1 | month | 0.021 | 0.066 |
| | | 3 | months | 0.035 | 0.074 |
| | | 6 | months | 0.039 | 0.088 |
| | 40° C. | 1 | month | 0.028 | 0.070 |
| | | 3 | months | 0.042 | 0.072 |
| | | 6 | months | 0.049 | 0.133 |
| | 60° C. | 10 | days | 0.053 | 0.159 |
| | | 1 | month | 0.098 | 0.194 |
| Example 3 | 0 day | | | 0.028 | 0.036 |
| | RT | 1 | month | 0.032 | 0.039 |
| | | 3 | months | 0.036 | 0.048 |
| | | 6 | months | 0.048 | 0.068 |
| | 40° C. | 1 | month | 0.041 | 0.052 |
| | | 3 | months | 0.055 | 0.087 |
| | | 6 | months | 0.058 | 0.126 |
| | 60° C. | 10 | days | 0.044 | 0.157 |
| | | 1 | month | 0.095 | 0.205 |
| Example 4 | 0 day | | | 0.030 | 0.043 |
| | RT | 1 | month | 0.039 | 0.058 |
| | | 3 | months | 0.042 | 0.068 |
| | | 6 | months | 0.055 | 0.086 |
| | 40° C. | 1 | month | 0.047 | 0.086 |
| | | 3 | months | 0.066 | 0.102 |
| | | 6 | months | 0.083 | 0.196 |
| | 60° C. | 10 | days | 0.059 | 0.203 |
| | | 1 | month | 0.125 | 0.325 |
| Example 9 | 0 day | | | 0.02 | 0.02 |
| | RT | 1 | month | 0.02 | 0.052 |
| | | 3 | months | 0.02 | 0.068 |
| | | 6 | months | 0.03 | 0.067 |
| | 40° C. | 1 | month | 0.02 | 0.055 |
| | | 3 | months | 0.03 | 0.06 |
| | | 6 | months | 0.05 | 0.124 |
| | 60° C. | 10 | days | 0.042 | 0.141 |
| | | 1 | month | 0.087 | 0.179 |
| Example 12 | 0 day | | | 0.02 | 0.02 |
| | RT | 1 | month | 0.03 | 0.055 |
| | | 3 | months | 0.03 | 0.056 |
| | | 6 | months | 0.02 | 0.077 |
| | 40° C. | 1 | month | 0.03 | 0.066 |
| | | 3 | months | 0.03 | 0.082 |
| | | 6 | months | 0.07 | 0.129 |
| | 60° C. | 10 | days | 0.061 | 0.199 |
| | | 1 | month | 0.091 | 0.225 |
| Example 13 | 0 day | | | 0.019 | 0.049 |
| | RT | 1 | month | 0.020 | 0.059 |
| | | 3 | months | 0.023 | 0.062 |
| | | 6 | months | 0.033 | 0.069 |
| | 40° C. | 1 | month | 0.028 | 0.057 |
| | | 3 | months | 0.033 | 0.070 |
| | | 6 | months | 0.059 | 0.096 |
| | 60° C. | 10 | days | 0.036 | 0.115 |
| | | 1 | month | 0.068 | 0.186 |
| Example 14 | 0 day | | | 0.019 | 0.055 |
| | RT | 1 | month | 0.022 | 0.066 |
| | | 3 | months | 0.021 | 0.063 |
| | | 6 | months | 0.038 | 0.072 |
| | 40° C. | 1 | month | 0.031 | 0.068 |
| | | 3 | months | 0.035 | 0.075 |
| | | 6 | months | 0.068 | 0.103 |
| | 60° C. | 10 | days | 0.04 | 0.159 |
| | | 1 | month | 0.071 | 0.201 |
| Example 16 | 0 day | | | 0.021 | 0.134 |
| | RT | 1 | month | 0.019 | 0.096 |
| | | 3 | months | 0.025 | 0.099 |
| | | 6 | months | 0.033 | 0.094 |
| | 40° C. | 1 | month | 0.031 | 0.087 |
| | | 3 | months | 0.042 | 0.101 |
| | | 6 | months | 0.058 | 0.121 |
| | 60° C. | 10 | days | 0.033 | 0.106 |
| | | 1 | month | 0.057 | 0.151 |
| Example 17 | 0 day | | | 0.020 | 0.153 |
| | RT | 1 | month | 0.022 | 0.106 |
| | | 3 | months | 0.026 | 0.116 |
| | | 6 | months | 0.035 | 0.123 |
| | 40° C. | 1 | month | 0.032 | 0.089 |
| | | 3 | months | 0.046 | 0.126 |
| | | 6 | months | 0.072 | 0.146 |
| | 60° C. | 10 | days | 0.055 | 0.115 |
| | | 1 | month | 0.089 | 0.198 |

Conclusion: compared with comparative examples 3 and 4, the pharmaceutical formulations of Examples 1, 3, 4, 9, 12, 13, 14, 16, and 17 of the present disclosure were stable when stored under both accelerated conditions and room temperature conditions, with a low content of maximum single impurity and/or a low content of total impurities.

### 4. Pharmacokinetic Study

Experimental objective: to study the differences in blood concentration and tissue distribution of posaconazole over time after intravenously administering to SD rats the pharmaceutical formulation prepared in Example 5 and the marketed posaconazole injection formulation (Jiangsu Aosaikang Pharmaceutical Co., Ltd., batch number: G220603), and estimate the pharmacokinetic parameters of posaconazole to compare the differences between the two injections.

Method: 24 male SD rats were randomly divided into 2 groups, with 12 animals in each group. Group 1 received 30 mg/kg of the pharmaceutical formulation prepared in Example 5 via tail vein injection, and Group 2 received 30 mg/kg of the marketed posaconazole injection formulation via tail vein injection. The animals were euthanized at 0.5 h, 1 h, 2 h, 4 h, 8 h, and 12 h after administration, and the blood and tissues were collected from the heart, liver, spleen, lung, and kidney. Whole blood was centrifuged as plasma samples, the tissues were homogenized and analyzed to obtain the drug concentrations, and pharmacokinetic parameters were fitted using the WinNonLin non-compartmental model.

### Detection Method

Liquid chromatography system: A complete set of Shimadzu liquid chromatography system including degasser (model DGU-20A5R), controller (CBM-20A), autosampler (SIL-30AC), column oven (CTO-30A), and pump (LC-30AD).
Liquid chromatography column: Phenomenex Omega C18, 50× 2.1 mm, 1.7 μm Column temperature: 40° C.
Autosampler temperature: 4° C.
Mobile phase A: 0.1% formic acid aqueous solution
Mobile phase B: acetonitrile
Cleaning mode: cleaning before and after sample injection, cleaning the pump→cleaning the port
Cleaning time: 1 second
Dipping time: 1 second
Needle Wash R3: acetonitrile/methanol/isopropanol/water (1:1:1:1)
Needle Wash R0: 50% methanol
Needle Wash volume and speed: 500 μL, 35 μL/s
Mode: Binary high-pressure gradient
Total flow rate: 0.400 mL/min
Pump B concentration: 50.0%

| Time (min) | Unit | Command | Value |
|---|---|---|---|
| 1.00 | Column oven | CTO.RVL | 1 |
| 2.00 | Column oven | CTO.RVL | 0 |
| 2.10 | Pump | B.Conc | 100 |
| 2.50 | Pump | B.Conc | 100 |
| 2.60 | Pump | B.Conc | 50 |
| 3.50 | Controller | Stop | |

Injection volume: 10 μL
Ionization mode: ESI, analysis mode: MRM+

| Parameter | Value | |
|---|---|---|
| Interface voltage | 3.5 | kV |
| Interface temperature | 300° | C. |
| DL temperature | 250° | C. |
| Atomizing gas flow rate | 3 | L/min |
| Heating gas flow rate | 10 | L/min |
| Heating block temperature | 400° | C. |
| Drying gas flow rate | 10 | L/min |

The experimental results were shown in Table 6 to Table 12.

TABLE 6
Area under the drug-time curve (AUC) in tissues and plasma

| | AUC (μg/mL*h or μg/g*h) | | | | | |
|---|---|---|---|---|---|---|
| Group | Heart | Liver | Spleen | Lung | Kidney | Plasma |
| Group 1-Example 5 | 341 | 752 | 231 | 254 | 288 | 95 |
| Group 2-Posaconazole | 299 | 703 | 235 | 234 | 303 | 83 |
| injection (marketed) | | | | | | |

TABLE 7
Average drug concentration in plasma (μg/mL)

| Time | Concentration (μg/mL) | |
|---|---|---|
| (h) | Group 1 (Example 5) | Posaconazole injection (marketed) |
| 0.5 | 11.658 | 10.073 |
| 1 | 10.572 | 9.197 |
| 2 | 9.646 | 8.095 |
| 4 | 8.217 | 7.627 |
| 8 | 7.562 | 6.218 |
| 12 | 7.317 | 6.903 |

TABLE 8
Average drug concentration in the heart (μg/g)

| Time | Concentration (μg/g) | |
|---|---|---|
| (h) | Group 1 (Example 5) | Posaconazole injection (marketed) |
| 0.5 | 43.050 | 37.154 |
| 1 | 41.544 | 40.359 |
| 2 | 29.880 | 33.562 |
| 4 | 24.471 | 25.659 |
| 8 | 32.767 | 21.261 |
| 12 | 24.853 | 23.780 |

TABLE 9
Average drug concentration in the liver (μg/g)

| Time | Concentration (μg/g) | |
|---|---|---|
| (h) | Group 1 (Example 5) | Posaconazole injection (marketed) |
| 0.5 | 91.363 | 78.405 |
| 1 | 93.952 | 74.381 |
| 2 | 73.237 | 72.239 |
| 4 | 66.337 | 64.705 |
| 8 | 61.118 | 52.640 |
| 12 | 52.910 | 57.051 |

TABLE 10
Average drug concentration in the spleen (μg/g)

| Time | Concentration (μg/g) | |
|---|---|---|
| (h) | Group 1 (Example 5) | Posaconazole injection (marketed) |
| 0.5 | 26.000 | 27.698 |
| 1 | 26.819 | 23.717 |
| 2 | 24.166 | 21.302 |
| 4 | 18.941 | 22.497 |
| 8 | 17.825 | 17.682 |
| 12 | 20.211 | 19.917 |

TABLE 11
Average drug concentration in the lung (μg/g)

| Time | Concentration (μg/g) | |
|---|---|---|
| (h) | Group 1 (Example 5) | Posaconazole injection (marketed) |
| 0.5 | 28.639 | 23.691 |
| 1 | 26.225 | 24.527 |
| 2 | 25.475 | 22.424 |
| 4 | 21.229 | 20.816 |
| 8 | 21.309 | 18.891 |
| 12 | 20.150 | 18.953 |

TABLE 12
Average drug concentration in the kidney (μg/g)

| Time | Concentration (μg/g) | |
|---|---|---|
| (h) | Group 1 (Example 5) | Posaconazole injection (marketed) |
| 0.5 | 28.606 | 31.945 |
| 1 | 29.001 | 30.974 |
| 2 | 29.278 | 28.125 |
| 4 | 24.180 | 29.168 |
| 8 | 23.755 | 23.218 |
| 12 | 23.924 | 24.395 |

Conclusion: the pharmaceutical formulation prepared in Example 5 and the marketed posaconazole injection formulation showed the substantially same distribution in the plasma and tissues after being administered intravenously to SD rats at the same dose of 30 mg/kg.

### 5. Hemolysis Study

Objective: to determine the safety of the pharmaceutical formulation prepared in Example 5 by observing the hemolysis effect in vitro of the pharmaceutical formulation prepared in Example 5 and comparing it with the marketed posaconazole injection, and to provide data support for its clinical applications.

Method: After anesthesia, blood was collected from the heart of New Zealand white rabbits, and prepared into 2% red blood cell suspensions, to which different volumes of the test samples were added, i.e., the pharmaceutical formulation prepared in Example 5 or the marketed posaconazole injection (Jiangsu Aosaikang Pharmaceutical Co., Ltd., G220603) (both at a concentration of 1.5 mg/mL, the dilution solvent: 5% glucose injection solution) as well as 0.9% sodium chloride injection solution. The 0.9% sodium chloride injection solution was used as a negative control and the sterilized water for injection was used as a positive control. After mixing, the test tubes were placed in a thermostat at 37° C.±0.5° C. for incubation and photographed, and then observed and photographed at 15 min, 30 min, 45 min, 1 h, 2 h, and 3 h after incubation, respectively. After 3 h of incubation and photographing, the test tubes were removed smoothly from the thermostat, and centrifuged at 1000 r/min for 15 min for further observation and photographing, to observe the hemolytic or coagulant effect of the pharmaceutical formulation prepared in Example 5 and the marketed posaconazole injection on the red blood cells of the rabbits. The results were shown in Table 13.

TABLE 13
In vitro hemolysis experiment

| | | | | | | Marketed Posaconazole | | | | | | |
|---|---|---|---|---|---|---|---|---|---|---|---|---|
| | Example 5 | | | | | injection | | | | | Negative | Positive |
| Tube No. | 1 | 2 | 3 | 4 | 5 | 6 | 7 | 8 | 9 | 10 | 11 | 12 |
| 2% red blood cell | 2.5 | 2.5 | 2.5 | 2.5 | 2.5 | 2.5 | 2.5 | 2.5 | 2.5 | 2.5 | 2.5 | 2.5 |
| suspension (ml) | | | | | | | | | | | | |
| 0.9% sodium | 2.0 | 2.1 | 2.2 | 2.3 | 2.4 | 2.0 | 2.1 | 2.2 | 2.3 | 2.4 | 2.5 | 0 |
| chloride injection | | | | | | | | | | | | |
| solution (ml) | | | | | | | | | | | | |
| Sterilized water for | 0 | 0 | 0 | 0 | 0 | 0 | 0 | 0 | 0 | 0 | 0 | 2.5 |
| injection (ml) | | | | | | | | | | | | |
| Test sample (ml) | 0.5 | 0.4 | 0.3 | 0.2 | 0.1 | 0.5 | 0.4 | 0.3 | 0.2 | 0.1 | 0 | 0 |

### Results

After 0.9% sodium chloride injection solution was added to the No. 11 negative control tube, the red blood cells in this test tube began to sink over time, and sank completely after 3 hours, and the supernatant was colorless and clear. After appropriate shaking, the sunken red blood cells were re-dispersed. After the re-dispersed solution was centrifuged, the supernatant of the solution was colorless and clear, indicating that hemolysis and coagulation phenomena did not occur.

After the sterilized water for injection was added to the No. 12 positive control tube, the solution in this test tube showed a clear red color immediately, with no red blood cell precipitation at the bottom of the tube. After 3 hours, the supernatant showed a clear red color, and after centrifugation, the solution still showed a clear red color, indicating that hemolysis had occurred.

After different volumes of the marketed posaconazole injection (1.5 mg/mL) were added to the tubes Nos. 6-10, the red blood cells in these test tubes began to sink over time and sank completely after 3 hours. The supernatants were colorless and clear. After appropriate shaking, the sunken red blood cells were re-dispersed. After the re-dispersed solutions were centrifuged, the supernatants of the solutions were colorless and clear, indicating that the hemolysis and coagulation phenomena did not occur.

After different volumes of the pharmaceutical formulation prepared in Example 5 (1.5 mg/mL) were added to the tubes Nos. 1-5, the red blood cells in these test tubes began to sink over time and sank completely after 3 hours. The supernatants were colorless and clear. After appropriate shaking, the sunken red blood cells were re-dispersed. After the re-dispersed solutions were centrifuged, the supernatants of the solutions were colorless and clear, indicating that hemolysis and coagulation phenomena did not occur.

Conclusion: in summary, under the conditions of this experiment, the reference formulation (the marketed posaconazole injection) and the pharmaceutical formulation prepared in Example 5 had no hemolytic or coagulant effect on the red blood cells of the rabbits at a concentration of 1.5 mg/mL, and the results of the hemolysis test in vitro were negative.

1. A pharmaceutical composition, comprising posaconazole or a pharmaceutically acceptable salt thereof, a good solvent, a stabilizer, a surfactant, a nanocrystal inducer and optionally a pH regulator, wherein the pharmaceutical composition is free of cyclodextrin and derivatives of cyclodextrin.

2. The pharmaceutical composition according to claim 1, wherein
the good solvent is selected from the group consisting of anhydrous ethanol, propylene glycol, polyethylene glycol, dimethyl sulfoxide, N-methylpyrrolidone, N,N-dimethylacetamide, N,N-dimethylformamide, and any combination thereof; or
the good solvent is selected from the group consisting of a mixture of anhydrous ethanol and polyethylene glycol, a mixture of propylene glycol and polyethylene glycol, dimethyl sulfoxide, N-methylpyrrolidone, N,N-dimethylacetamide, and N,N-dimethylformamide; or
the good solvent is selected from the group consisting of N,N-dimethylacetamide, N,N-dimethylformamide, dimethyl sulfoxide, a mixture of propylene glycol and polyethylene glycol, and a mixture of anhydrous ethanol and polyethylene glycol; or
the good solvent is selected from a mixture of anhydrous ethanol and polyethylene glycol 400, a mixture of anhydrous ethanol and polyethylene glycol 300, a mixture of anhydrous ethanol and polyethylene glycol 200, a mixture of propylene glycol and polyethylene glycol 400, a mixture of propylene glycol and polyethylene glycol 300, or a mixture of propylene glycol and polyethylene glycol 200; or
the good solvent is selected from a mixture of anhydrous ethanol and polyethylene glycol 400, a mixture of anhydrous ethanol and polyethylene glycol 300, or a mixture of anhydrous ethanol and polyethylene glycol 200.

3. The pharmaceutical composition according to claim 1, wherein
the stabilizer is selected from the group consisting of ascorbyl palmitate, sodium metabisulfite, α-tocopherol, butylated droxyanisole, butylated hydroxytoluene, methionine, proline, glycine, and any combination thereof; or
the stabilizer is selected from the group consisting of ascorbyl palmitate, α-tocopherol, butylated droxyanisole, butylated hydroxytoluene, methionine, proline, glycine, and any combination thereof; or
the stabilizer is selected from the group consisting of methionine, α-tocopherol, ascorbyl palmitate, butylated droxyanisole, butylated hydroxytoluene, and any combination thereof; or
the stabilizer is selected from the group consisting of α-tocopherol, ascorbyl palmitate, butylated droxyanisole, butylated hydroxytoluene, and any combination thereof; or
the stabilizer is selected from the group consisting of α-tocopherol, ascorbyl palmitate, and a combination of butylated droxyanisole and butylated hydroxytoluene.

4. The pharmaceutical composition according to claim 2, wherein
the stabilizer is selected from the group consisting of ascorbyl palmitate, sodium metabisulfite, α-tocopherol, butylated droxyanisole, butylated hydroxytoluene, methionine, proline, glycine, and any combination thereof; or
the stabilizer is selected from the group consisting of ascorbyl palmitate, α-tocopherol, butylated droxyanisole, butylated hydroxytoluene, methionine, proline, glycine, and any combination thereof; or
the stabilizer is selected from the group consisting of methionine, α-tocopherol, ascorbyl palmitate, butylated droxyanisole, butylated hydroxytoluene, and any combination thereof; or
the stabilizer is selected from the group consisting of α-tocopherol, ascorbyl palmitate, butylated droxyanisole, butylated hydroxytoluene, and any combination thereof; or
the stabilizer is selected from the group consisting of α-tocopherol, ascorbyl palmitate, and a combination of butylated droxyanisole and butylated hydroxytoluene.

5. The pharmaceutical composition according to claim 1, wherein
the surfactant is selected from the group consisting of glycocholic acid, glycocholate, deoxycholic acid, deoxycholate, tauroursodeoxycholic acid, tauroursodeoxycholate, taurocholic acid, taurocholate, taurodeoxycholic acid, taurodeoxycholate, polyoxyl 15 hydroxystearate, polyethylene glycol vitamin E succinate, and any combination thereof; or
the surfactant is selected from the group consisting of polyethylene glycol vitamin E succinate, deoxycholic acid, deoxycholate, glycocholic acid, glycocholate, taurodeoxycholic acid, taurodeoxycholate, and any combination thereof; or
the surfactant is one or two selected from glycocholic acid, sodium glycocholate, or polyethylene glycol vitamin E succinate.

6. The pharmaceutical composition according to claim 2, wherein
the surfactant is selected from the group consisting of glycocholic acid, glycocholate, deoxycholic acid, deoxycholate, tauroursodeoxycholic acid, tauroursodeoxycholate, taurocholic acid, taurocholate, taurodeoxycholic acid, taurodeoxycholate, polyoxyl 15 hydroxystearate, polyethylene glycol vitamin E succinate, and any combination thereof; or
the surfactant is selected from the group consisting of polyethylene glycol vitamin E succinate, deoxycholic acid, deoxycholate, glycocholic acid, glycocholate, taurodeoxycholic acid, taurodeoxycholate, and any combination thereof; or
the surfactant is one or two selected from glycocholic acid, sodium glycocholate, or polyethylene glycol vitamin E succinate.

7. The pharmaceutical composition according to claim 3, wherein
the surfactant is selected from the group consisting of glycocholic acid, glycocholate, deoxycholic acid, deoxycholate, tauroursodeoxycholic acid, tauroursodeoxycholate, taurocholic acid, taurocholate, taurodeoxycholic acid, taurodeoxycholate, polyoxyl 15 hydroxystearate, polyethylene glycol vitamin E succinate, and any combination thereof; or
the surfactant is selected from the group consisting of polyethylene glycol vitamin E succinate, deoxycholic acid, deoxycholate, glycocholic acid, glycocholate, taurodeoxycholic acid, taurodeoxycholate, and any combination thereof; or
the surfactant is one or two selected from glycocholic acid, sodium glycocholate, or polyethylene glycol vitamin E succinate.

8. The pharmaceutical composition according to claim 1, wherein
the nanocrystal inducer is selected from the group consisting of pamoic acid, hydroxynaphthoic acid, propyl gallate, benzoic acid, and any combination thereof; or
the nanocrystal inducer is selected from pamoic acid and/or hydroxynaphthoic acid; or
the nanocrystal inducer is selected from pamoic acid, 3-hydroxy-1-naphthoic acid, or 2-hydroxy-3-naphthoic acid; or
the nanocrystal inducer is pamoic acid.

9. The pharmaceutical composition according to claim 2, wherein
the nanocrystal inducer is selected from the group consisting of pamoic acid, hydroxynaphthoic acid, propyl gallate, benzoic acid, and any combination thereof; or
the nanocrystal inducer is selected from pamoic acid and/or hydroxynaphthoic acid; or
the nanocrystal inducer is selected from pamoic acid, 3-hydroxy-1-naphthoic acid, or 2-hydroxy-3-naphthoic acid; or
the nanocrystal inducer is pamoic acid.

10. The pharmaceutical composition according to claim 3, wherein
the nanocrystal inducer is selected from the group consisting of pamoic acid, hydroxynaphthoic acid, propyl gallate, benzoic acid, and any combination thereof; or
the nanocrystal inducer is selected from pamoic acid and/or hydroxynaphthoic acid; or
the nanocrystal inducer is selected from pamoic acid, 3-hydroxy-1-naphthoic acid, or 2-hydroxy-3-naphthoic acid; or
the nanocrystal inducer is pamoic acid.

11. The pharmaceutical composition according to claim 5, wherein
the nanocrystal inducer is selected from the group consisting of pamoic acid, hydroxynaphthoic acid, propyl gallate, benzoic acid, and any combination thereof; or
the nanocrystal inducer is selected from pamoic acid and/or hydroxynaphthoic acid; or
the nanocrystal inducer is selected from pamoic acid, 3-hydroxy-1-naphthoic acid, or 2-hydroxy-3-naphthoic acid; or
the nanocrystal inducer is pamoic acid.

12. The pharmaceutical composition according to claim 1, wherein
the pH regulator is selected from the group consisting of sodium hydroxide, potassium hydroxide, tromethamine, meglumine, and any combination thereof; or
the pH regulator is selected from the group consisting of sodium hydroxide and potassium hydroxide.

13. The pharmaceutical composition according to claim 2, wherein
the pH regulator is selected from the group consisting of sodium hydroxide, potassium hydroxide, tromethamine, meglumine, and any combination thereof; or
the pH regulator is selected from the group consisting of sodium hydroxide and potassium hydroxide.

14. The pharmaceutical composition according to claim 3, wherein
the pH regulator is selected from the group consisting of sodium hydroxide, potassium hydroxide, tromethamine, meglumine, and any combination thereof; or
the pH regulator is selected from the group consisting of sodium hydroxide and potassium hydroxide.

15. The pharmaceutical composition according to claim 5, wherein
the pH regulator is selected from the group consisting of sodium hydroxide, potassium hydroxide, tromethamine, meglumine, and any combination thereof; or
the pH regulator is selected from the group consisting of sodium hydroxide and potassium hydroxide.

16. The pharmaceutical composition according to claim 8, wherein
the pH regulator is selected from the group consisting of sodium hydroxide, potassium hydroxide, tromethamine, meglumine, and any combination thereof; or
the pH regulator is selected from the group consisting of sodium hydroxide and potassium hydroxide.

17. The pharmaceutical composition according to claim 1, wherein
the pharmaceutical composition forms a nanosuspension after being diluted with an antisolvent, wherein particles in the nanosuspension have a particle size ranging from about 50 nm to about 300 nm or from about 50 nm to about 200 nm, and/or the nanosuspension has a pH ranging from about 3.5 to about 8.0 or from about 4.0 to about 6.0, and the antisolvent is selected from the group consisting of sterilized water for injection, a 5% aqueous glucose solution, a 10% aqueouglucose s solution, a 0.45% aqueous sodium chloride solution, a 0.9% aqueous sodium chloride solution, a 5% aqueous glucose and 0.45% aqueous sodium chloride solution, a 5% aqueous glucose and 0.9% aqueous sodium chloride solution, and a 5% aqueous glucose and 20 mEq aqueous sodium chloride solution.

18. The pharmaceutical composition according to claim 1, wherein
the pharmaceutical composition comprises about 1 g to about 10 g of posaconazole in the form of a free base, about 0.01 g to about 1.0 g of the stabilizer, about 0.1 g to about 7.0 g of the surfactant, about 0.2 g to about 4.0 g of the nanocrystal inducer, and about 0 g to about 1.0 g of the pH regulator, based on 100 ml of a total volume of the good solvent; or
the pharmaceutical composition comprises about 2.0 g to about 3.0 g of posaconazole in the form of a free base, about 0.1 g to about 0.2 g of the stabilizer, about 0.2 g to 3.0 g of the surfactant, about 0.2 g to about 1.0 g of the nanocrystal inducer, and about 0.1 g to 0.5 g of the pH regulator, based on 100 ml of a total volume of the good solvent.

19. The pharmaceutical composition according to claim 18, wherein a concentration of posaconazole in the form of a free base in the nanosuspension is about 0.5 mg/ml to about 5 mg/ml, or about 1 mg/ml to about 2 mg/ml.

20. A method for treating and preventing fungal infections in a patient, comprising administering the pharmaceutical composition according to claim 1 to the patient, wherein the fungal infections are selected from the group consisting of oropharyngeal or esophageal candidiasis; refractory oropharyngeal or esophageal candidiasis; invasive aspergillosis, candidiasis, fusaridiosis, scedosporiosis, infections caused by dimorphic fungi, zygomycosis, and invasive infections caused by rare fungi and yeasts; invasive fungal diseases in patients who are not responsive or intolerant to other therapies; candidiasis and invasive mycotic infections in patients undergoing intensive chemotherapies and/or radiotherapies due to hematological malignancies, bone marrow or peripheral stem cell transplantation pretreatment regimens, and in patients receiving combined immunosuppressive therapies for the treatment of acute or chronic graft-versus-host disease or for the prevention of solid organ transplant rejection; Chagas's disease; and leishmaniasis.
